(19) United States
(12) Patent Application Publication
(10) Pub. No.: US 2005/0063920 A1
(43) Pub. Date: Mar. 24, 2005

(54) Oral composition

(75) Inventors: Han Ting Chang, Livermore, CA (US); Dominique Charmot, Campbell, CA (US); David Duncalf, Bebington (GB); Oleg Kolosov, San Jose, CA (US); Victor Nava-Salgado, San Jose, CA (US); Son Hoai Nguyen, Santa Clara, CA (US); Miroslav Petro, San Jose, CA (US); Steven Paul Rannard, Bebington (GB)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Unilever Home & Personal Care USA, Division of Conopco, Inc.

(21) Appl. No.: 10/666,487

(22) Filed: Sep. 19, 2003

(51) Int. Cl. A61K 9/68; A61K 7/16

(52) U.S. Cl. 424/48; 424/49

(57) ABSTRACT

Oral care composition comprising a cationic polymer obtainable by copolymerising a mixture of comonomers, said mixture comprising (ar-vinylbenzyl) trimethylammonium chloride, 2-acrylamido-2-methyl-1-propanesulfonic acid and polyethyleneglycol methylethermethacrylate, said composition being in the form of any one of a toothpaste, gel, foam, chewing gum, deformable strip or mouthwash and being suitable for use in the oral cavity.

The present invention relates to an oral composition comprising a polymer which is delivered to the oral surfaces during toothbrushing.

We have found that there exists a range of polymers which are delivered more effectively to the oral surfaces during brushing. Accordingly, these polymers provide a useful tool for the delivery of active substances for the treatment or prevention of oral care related conditions such as gingivitis, caries, tartar, oral malodour, etc.

Accordingly the present invention provides an oral care composition comprising a cationic polymer obtainable by copolymerising a mixture of comonomers, said mixture comprising (ar-vinylbenzyl) trimethylammonium chloride, 2-acrylamido-2-methyl-1-propanesulfonic acid and polyethyleneglycol methylethermethacrylate,

said composition being in the form of any one of a toothpaste, gel, foam, chewing gum, deformable strip or mouthwash and being suitable for use in the oral cavity.

The polymerisation mixture preferably comprises from 5 to 95 mol % (ar-vinylbenzyl) trimethylammonium chloride, preferably from 30 to 65 mol % and especially 45 mol %.

The polymerisation mixture preferably comprises from 5 to 95 mol % polyethyleneglycol methylethermethacrylate, preferably from 30 to 65 mol % and especially 40 mol %.

The polymerisation mixture preferably comprises from 5 to 50 mol % 2-acrylamido-2-methyl-1-propanesulfonic acid, preferably from 10 to 25 mol % and especially 15 mol %.

The polymer according to the invention is preferably present at from 0.01 to 10% by weight of the composition. Preferably, in an amount ranging from 0.05 to 5% by weight of the composition.

The composition according to the invention may also comprise a halogenated hydroxydiphenyl ether compound, more preferably 2′,4,4′-trichloro-2-hydroxy-diphenyl ether, hereinafter known as triclosan. Preferably the halogenated hydroxydiphenyl ether is present at from 0.01 to 0.5% by weight of the composition. A further preferred group of antimicrobial substances are the parahydroxybenzoic acid esters, also known as parabens, and their structural analogues. Preferred parabens are the medium chain length parabens such as hexyl, heptyl, octyl, nonyl and decyl parabens. Most preferred is the n-octyl paraben. The polymer according to the invention is particularly effective in delivering these parahydroxy benzoate ester antimicrobial agents.

The composition according to the invention may also comprise a divalent metal salt. Preferably, the divalent metal salt is a salt selected from the group consisting of zinc- and stannous salts such as zinc citrate, zinc sulphate, zinc glycinate, sodium zinc citrate, stannous pyrophosphate and mixtures thereof. The preferable divalent metal salt is zinc citrate.

Suitably, the amount of divalent metal salt ranges from 0.01 to 10% by weight of the composition, preferably from 0.05 to 5% by weight, more preferably from 0.1 to 2% by weight and especially preferably from 0.3 to 0.9% by weight of the composition.

The oral composition according to the invention comprise further ingredients which are common in the art, such as:
antimicrobial agents, e.g. chlorhexidine, sanguinarine extract, metronidazole, quaternary ammonium compounds, such as cetylpyridinium chloride; bis-guanides, such as chlorhexidine digluconate, hexetidine, octenidine, alexidine; and halogenated bisphenolic compounds, such as 2,2′ methylenebis-(4-chloro-6-bromophenol);
anti-inflammatory agents such as ibuprofen, flurbiprofen, aspirin, indomethacin etc.;
anti-caries agents such as sodium- and stannous fluoride, aminefluorides, sodium monofluorophosphate, sodium trimeta phosphate and casein;
plaque buffers such as urea, calcium lactate, calcium glycerophosphate and strontium polyacrylates;
vitamins such as Vitamins A, C and E;
plant extracts;
desensitising agents, e.g. potassium citrate, potassium chloride, potassium tartrate, potassium bicarbonate, potassium oxalate, potassium nitrate and strontium salts;
anti-calculus agents, e.g. alkali-metal pyrophosphates, hypophosphite-containing polymers, organic phosphonates and phosphocitrates etc.;
biomolecules, e.g. bacteriocins, antibodies, enzymes, etc.;
flavours, e.g. peppermint and spearmint oils;
proteinaceous materials such as collagen;
preservatives;
opacifying agents;
colouring agents;
pH-adjusting agents;
sweetening agents;
pharmaceutically acceptable carriers, e.g. starch, sucrose, water or water/alcohol systems etc.;
surfactants, such as anionic, nonionic, cationic and zwitterionic or amphoteric surfactants;
particulate abrasive materials such as silicas, aluminas, calcium carbonates, dicalciumphosphates, calcium pyrophosphates, hydroxyapatites, trimetaphosphates, insoluble hexametaphosphates and so on, including agglomerated particulate abrasive materials, usually in amounts between 3 and 60% by weight of the oral care composition. Preferred abrasives are chalk and silica, more preferably fine ground natural chalk.

Humectants such as glycerol, sorbitol, propyleneglycol, xylitol, lactitol etc.;
binders and thickeners such as sodium carboxymethyl-cellulose, hydroxyethyl cellulose (Natrosol®), xanthan gum, gum arabic etc. as well as synthetic polymers such as polyacrylates and carboxyvinyl polymers such as Carbopol®;
polymeric compounds which can enhance the delivery of active ingredients such as antimicrobial agents can also be included;
buffers and salts to buffer the pH and ionic strength of the oral care composition; and
other optional ingredients that may be included are e.g. bleaching agents such as peroxy compounds e.g. potassium peroxydiphosphate, effervescing systems such as sodium bicarbonate/citric acid systems, colour change systems, and so on.

Liposomes may also be used to improve delivery or stability of active ingredients.

The oral compositions may be in any form common in the art, e.g. toothpaste, gel, mousse, aerosol, gum, lozenge, powder, cream, etc. and may also be formulated into systems for use in dual-compartment type dispensers.

The polymer according to the invention is capable of delivering itself to the oral surfaces during brushing. Preferably, in conjunction with a benefit agent selected from any of those included herein. Most preferable of these benefit agents are the antimicrobials, anti-caries agents, anti-tartar agents, anti-malodour agents and bleaching or tooth whitening agents.

In a second aspect the present invention provides a process for preparing an oral care composition according to any one of claims 1 to 5, comprising the steps of:
preparing a mixture of comonomers as defined in the first aspect of the invention in an ethanol/water diluent;
polymerising the mixture by heating it under inert gas in the presence of an initiator;
extracting the polymer so obtained and blending it with one or more oral care actives and/or excipients so as to produce an oral care composition which is in the form of any one of a toothpaste, gel, foam, chewing gum, deformable strip or mouthwash and which is suitable for use in the oral cavity.

Preferably, the monomers are mixed at about 20% by (w/v) in ethanol:water mixture of from 50:50 to 95:5, more preferably from 70:30 to 90:10 and most preferably 80:20.

Preferably, the initiator is AIBN and is added at from 0.1 to 5%, preferably from 0.5 to 2.0% and most preferably at 1.0% mol with respect to the total monmomers.

Preferably, the inert gas is argon.

Preferably, the heating step involves heating for up to 36, preferably up to 24 and most preferably for 18 hours at above 45° C., preferably more than 50° C. and most preferably at about 65° C.

The monomer mixture is then preferably cooled to room temperature.

The polymer is then preferably, diluted with ethanol:water of from 50:50 to 95:5, more preferably from 70:30 to 90:10 and most preferably 80:20 to bring the final concentration to about 10% (w/v).

Preferably the reaction is carried out in a well of a 96-well plate.

### EXAMPLE 1

Manufacture of Polymers

Manufacture of polymers is done by preparing a mixture of comonomers as defined in any one of claims 1 or 2 in an ethanol/water diluent and polymerising the mixture by heating it under inert gas in the presence of an initiator.

### EXAMPLE 2

Pig tongue was pre treated with 2 ml of saliva containing 2.0 g/l sodium lauryl sulfate for 1 hour and rinsed twice with distilled water (6 sec each rinsing). The pig tongue was then exposed to 2 ml of toothpaste slurry (a paste formulation containing 0.3% Triclosan and 1.0% hit polymer diluted 1:3 with distilled water) for 2 min after which time the pig tongue was rinsed with 2 ml distilled water 5 times, 6 sec each rinsing.

One millilitre of ethanol was used to extract the Triclosan from the pig tongue for 60 min and the extract was filtered before HPLC analysis.

The values for Triclosan delivery are relative to the blank which was designated the value of 1.0.

| | | | | | | | Delivery |
|---|---|---|---|---|---|---|---|
| Sample | Monomer 1 | mol % | Monomer 2 | mol % | Monomer 3 | mol % | of TCN |
| 1 | VBTMAC | 45 | AMMPSA | 15 | PEGMEMA* | 40 | 1.27 |
| 2 | VBTMAC | 45 | AMMPSA | 15 | PEGMEMA** | 40 | 1.23 |

PEGEMA* is 475K polyethylene glycol methylethermethacrylate
PEGEMA** is 2000K polyethylene glycol methylethermethacrylate
VBTMAC is (ar-vinylbenzyl) trimethylammonium chloride
AMMPSA is 2-acrylamido-2-methyl-1-propanesulfonic acid

1. Oral care composition comprising a cationic polymer obtainable by copolymerising a mixture of comonomers, said mixture comprising (ar-vinylbenzyl) trimethylammonium chloride, 2-acrylamido-2-methyl-1-propanesulfonic acid and polyethyleneglycol methylethermethacrylate,
said composition being in the form of any one of a toothpaste, gel, foam, chewing gum, deformable strip or mouthwash and being suitable for use in the oral cavity.

2. Oral care composition according to claim 1, wherein the polyethyleneglycol methylethermethacrylate comprises from 5 to 100 polyethylene glycol subunits, preferably from 6 to 50 and especially preferably from 8 to 35.

3. Process for preparing an oral care composition according to claim 1, comprising the steps of:
preparing a mixture of comonomers as defined in any one of claims 1 or 2 in an ethanol/water diluent;
polymerising the mixture by heating it under inert gas in the presence of an initiator;
extracting the polymer so obtained and blending it with one or more oral care actives and/or excipients so as to produce an oral care composition which is in the form of any one of a toothpaste, gel, foam, chewing gum, deformable strip or mouthwash and which is suitable for use in the oral cavity.
